(19) United States
(12) Patent Application Publication
(10) Pub. No.: US 2015/0209392 A1
(43) Pub. Date: Jul. 30, 2015

(54) Filaments Comprising a Microorganism and Method for Making Same

(71) Applicant: The Procter & Gamble Company, Cincinnati, OH (US)

(72) Inventors: Brian Xiaqing Song, Mason, OH (US); Robin Lynn McKiernan, Mason, OH (US); Sharon Anne Keegan, Springboro, OH (US)

(73) Assignee: The Procter & Gamble Company

(21) Appl. No.: 14/576,244

(22) Filed: Dec. 19, 2014

Related U.S. Application Data
(60) Provisional application No. 61/931,153, filed on Jan. 24, 2014.

(51) Int. Cl. A61K 35/66; A61L 15/42; A61L 15/36; A61K 35/74; A61K 35/76

(52) U.S. Cl. CPC A61K 35/66; A61K 35/74; A61K 35/76; A61L 15/36; A61L 15/42

(57) ABSTRACT

Filaments containing one or more filament-forming materials and one or more microorganisms, for example a labile microorganism, and method for making same are provided.

### FIELD OF THE INVENTION

The present invention relates to filaments comprising one or more microorganisms, for example a labile microorganism, and more particularly to filaments, for example meltblown and/or dry spun and/or spunbond rather than electrospun, and/or micron diameter (i.e., 1-100 micron diameter) rather than nano diameter filaments, comprising one or more filament-forming materials, such as a hydroxyl polymer, and one or more microorganisms, such as a probiotic, and method for making same.

### BACKGROUND OF THE INVENTION

Electrospun and/or nanofiber filaments comprising biological active agents, such as bacteria and viruses are known. In one example, the electrospun and/or nanofiber filaments of the prior art are made from non-hydroxyl polymer filament-forming polymers, such as polyvinylpyrrolidone, which is not a hydroxyl polymer. In another example, the electrospun and/or nanofiber filaments of the prior art do not sufficiently stabilize their microorganisms during and/or after spinning of the filaments. For example, several microorganisms in one known electrospun filament had their viabilities significantly reduced by the electrospinning process and in order to prevent loss of even more viability the electrospun filaments had to be stored at temperatures of −20° C. or below, which is not conducive for use by consumers of products made with such electrospun filaments.

Other known filaments comprising microorganisms fail to teach stabilizing the microorganisms within the filaments with or without stabilizing agents such that the microorganism exhibits less than a 3 log loss after being exposed to 30° C./30% relative humidity (“RH”) conditions for 28 days as measured according to the Viability/Count Test Method described herein. Current distribution of products in commerce containing for example probiotics is done in special packaging which is moisture impermeable and maintains the probiotic containing material in a low humidity state so as to minimize the loss of microorganisms. Also, such products are kept at relatively low temperatures and in some instances are refrigerated or even frozen, which also is not conducive for use by consumers of such products.

Accordingly, one problem of known filaments comprising microorganisms is that the filaments fail to sufficiently stabilize the microorganisms such that the microorganisms exhibit less than a 6.5 (56 days) and/or 2.7 (28 days) log viability loss and/or to contain a total level of at least 10³ CFU/g of at least one of the microorganisms after being exposed to 30° C./30% RH conditions for 28 and/or 56 days as measured according to the Viability/Count Test Method described herein and/or to dissolve, such as under conditions of intended use, and therefore release one or more microorganisms in less than 60 minutes as measured by the Dissolution Test Method described herein.

One problem associated with delivering microorganisms, such as probiotics via a solid delivery vehicle, such as a filament, is that the probiotics lose their viability and/or their count (total level of each microorganism) is significantly reduced during and/or after formation of the solid delivery vehicle, such as the filaments containing the probiotics.

Accordingly, there is a need for a filament that comprises one or more microorganisms, such as probiotics, that exhibits improved viability, for example as a result of improved stability, over known filaments comprising microorganisms.

### SUMMARY OF THE INVENTION

The present invention fulfills the need described above by providing a filament, such as a meltblown and/or dry spun and/or spunbond rather than electrospun, and/or micron diameter rather than nano diameter filament, comprising a microorganism that exhibits a viability and/or stability greater than known filaments comprising microorganisms.

One solution to the problem identified above is a filament comprising a filament-forming material and one or more microorganisms such that at least one of the microorganisms present in the filament exhibits less than a 6.5 (56 days) and/or 2.7 (28 days) log viability loss and/or a count of at least 10³ CFU/g of filament after being exposed to 30° C./30% RH conditions for 28 and/or 56 days as measured according to the Viability/Count Test Method described herein.

It has been unexpectedly found that filaments that contain one or more microorganisms of the present invention provide sufficient stability to the microorganisms to maintain their viability and/or count while present in the filament such that the microorganisms exhibit less than a 6.5 (56 days) and/or 2.7 (28 days) log viability loss upon exposure to 30° C./30% RH conditions for 28 and/or 56 days and which can be released from the filaments under conditions of intended use when the filament dissolves.

In one example of the present invention, a filament comprising a filament-forming material and one or more microorganisms, wherein at least one of the microorganisms exhibits less than a 2.7 log viability loss after being exposed to 30° C./30% RH conditions for 28 days as measured according to the Viability/Count Test Method, is provided.

In another example of the present invention, a filament comprising one or more filament-forming materials, one or more microorganisms, and one or more stabilizing agents wherein the filament exhibits a cross-section that comprises two or more microorganisms, is provided.

In still another example of the present invention, a filament comprising one or more filament-forming materials and one or more microorganisms, wherein the filament contains at least 10³ CFU/g and/or at least 10⁴ CFU/g and/or at least 10⁵ CFU/g and/or at least 10⁶ CFU/g and/or at least 10⁷ CFU/g and/or at least 10⁸ CFU/g of at least one of the microorganisms after being exposed to 30° C./30% RH conditions for 28 and/or 56 days as measured according to the Viability/Count Test Method described herein, is provided.

In even another example of the present invention, a filament comprising one or more filament-forming materials and one or more microorganisms, wherein the filament exhibits an average dissolution time of less than 60 minutes as measured according to the Dissolution Test Method described herein, is provided.

In still another example of the present invention, a filament comprising a filament-forming material and one or more microorganisms, wherein at least one of the microorganisms exhibits less than a 6.5 log viability loss after being exposed to 30° C./30% RH conditions for 56 days as measured according to the Viability/Count Test Method, is provided.

In another example, a disposable absorbent article, for example a feminine hygiene pad, pantiliner, tampon, sanitary napkin, adult incontinence pad, adult incontinence pant, diaper, baby pant, toddler pant, overnight pant, swim pant, and mixtures thereof, comprising one or more filaments according to the present invention, is provided.

Accordingly, the present invention provides novel filaments that comprise one or more microorganisms and a method for making same.

### BRIEF DESCRIPTION OF THE DRAWINGS

FIG. 1 is a schematic representation of an example of a filament according to the present invention;

FIG. 2 is a schematic representation of an example of a process for making a filament according to the present invention;

FIG. 3 is a schematic representation of an example of a die suitable for use in the process of the present invention;

FIG. 4 is a front elevation view of a set-up for the Dissolution Test Method;

FIG. 5 is a partial top view of FIG. 4; and

FIG. 6 is a side elevation view of FIG. 4.

### DETAILED DESCRIPTION OF THE INVENTION

### Definitions

“Filament” as used herein means an elongate particulate having a length greatly exceeding its diameter, i.e. a length to diameter ratio of at least about 10.

The filaments of the present invention may be spun from filament-forming compositions via suitable spinning process operations, such as meltblowing, dry spinning, and/or spunbonding.

The filaments of the present invention may be monocomponent and/or multicomponent. For example, the filaments may comprise bicomponent filaments. The bicomponent filaments may be in any form, such as side-by-side, core and sheath, islands-in-the-sea and the like.

The filaments of the present invention exhibit a length of greater than or equal to 5.08 cm (2 in.) and/or greater than or equal to 7.62 cm (3 in.) and/or greater than or equal to 10.16 cm (4 in.) and/or greater than or equal to 15.24 cm (6 in.).

Filaments are typically considered continuous or substantially continuous in nature. Filaments are relatively longer than fibers (which are less than 5.08 cm in length). Non-limiting examples of filaments include meltblown and/or spunbond filaments.

In one example, one or more fibers may be formed from a filament of the present invention, such as when the filaments are cut to shorter lengths (such as less than 5.08 cm in length). Thus, in one example, the present invention also includes a fiber made from a filament of the present invention, such as a fiber comprising one or more filament-forming materials and one or more microorganisms. Therefore, references to filament and/or filaments of the present invention herein also include fibers made from such filament and/or filaments unless otherwise noted. Fibers are typically considered discontinuous in nature relative to filaments, which are considered continuous in nature.

“Filament-forming composition” as used herein means a composition that is suitable for making a filament of the present invention such as by meltblowing, dry spinning, and/or spunbonding. The filament-forming composition comprises one or more filament-forming materials that exhibit properties that make them suitable for spinning into a filament. In one example, the filament-forming material comprises a polymer. In addition to one or more filament-forming materials, the filament-forming composition may comprise one or more additives. In addition, the filament-forming composition may comprise one or more polar solvents, such as water, into which one or more, for example all, of the filament-forming materials and/or one or more, for example all, of the microorganisms and any additional additives, such as stabilizing agents and antioxidants, are dissolved and/or dispersed.

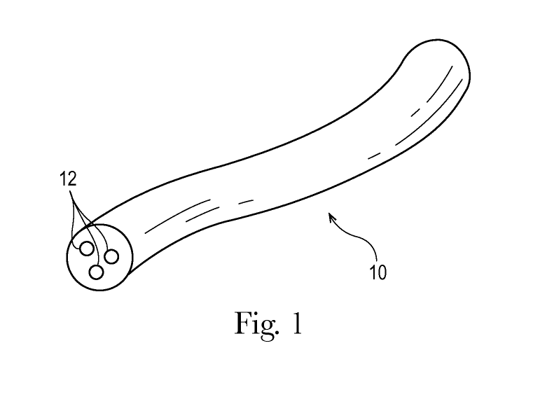

In one example as shown in FIG. 1 a filament 10 of the present invention made from a filament-forming composition comprising one or more filament-forming materials and one or more microorganisms of the present invention is such that one or more microorganisms 12, may be present in the filament rather than on the filament, such as a coating on an exterior surface of the filament. The total level of filament-forming materials and total level of microorganisms present in the filament-forming composition may be any suitable amount so long as the filaments of the present invention are produced therefrom. As is shown in FIG. 1, the cross-section of the filament may comprise two or more microorganisms.

In one example, one or more microorganisms may be present in the filament and one or more additional microorganisms may be present on a surface of the filament. In another example, a filament of the present invention may comprise one or more microorganisms that are present in the filament when originally made, but then are liberated from the filament when exposed to conditions of intended use of the filament.

“Filament-forming material” as used herein means a material, such as a polymer or monomers capable of producing a polymer that exhibits properties suitable for making a filament. In one example, the filament-forming material comprises one or more substituted polymers such as an anionic, cationic, zwitterionic, and/or nonionic polymer. In another example, the polymer may comprise a hydroxyl polymer, such as a polyvinyl alcohol (“PVOH”) and/or a polysaccharide, such as starch and/or a starch derivative, such as an ethoxylated starch and/or acid-thinned starch. In yet another example, the filament-forming material is a polar solvent-soluble material.

“Stabilizing agent” as used herein means a material that improves the viability of the microorganism, for example by preventing and/or mitigating the dehydration of the microorganisms during and/or after the formation of the filament containing the microorganism.

“Additive” as used herein means any material present in the filament of the present invention that is not a filament-forming material or a microorganism. In one example, an additive comprises a processing aid. In still another example, an additive comprises a filler. In one example, an additive comprises any material present in the filament that its absence from the filament would not result in the filament losing its filament structure, in other words, its absence does not result in the filament losing its solid form.

In one example, an additive comprises a stabilizing agent, for example a carbohydrate and/or protein, which provides the microorganism a stabilized environment within the filament.

In another example, an additive comprises an antioxidant.

In another example, an additive comprises a plasticizer for the filament. The filaments of the present invention may comprise one or more plasticizers. When present, the plasticizers may be present in the filament at a level of from about 0.01% to about 5% and/or from about 0.05% to about 3% and/or from about 0.05% to about 1% and/or from about 0.1 to about 0.5% by weight on a dry filament basis.

Non-limiting examples of suitable plasticizers for the present invention include polyols, copolyols, polycarboxylic acids, polyesters and dimethicone copolyols. Examples of useful polyols include, but are not limited to, glycerin, diglycerin, propylene glycol, ethylene glycol, butylene glycol, pentylene glycol, cyclohexane dimethanol, hexanediol, 2,2,4-trimethylpentane-1,3-diol, polyethylene glycol (200-600), pentaerythritol, sugar alcohols such as sorbitol, manitol, lactitol and other mono- and polyhydric low molecular weight alcohols (e.g., C2-C8 alcohols).

In one example, the plasticizer includes glycerin and/or propylene glycol and/or glycerol derivatives such as propoxylated glycerol. In still another example, the plasticizer is selected from the group consisting of glycerin, ethylene glycol, polyethylene glycol, propylene glycol, glycidol, urea, sorbitol, xylitol, maltitol, ethylene bisformamide, and mixtures thereof

In another example, an additive comprises a crosslinking agent suitable for crosslinking one or more of the filament-forming materials present in the filaments of the present invention. In one example, the crosslinking agent comprises a crosslinking agent capable of crosslinking hydroxyl polymers together, for example via their hydroxyl moieties. Non-limiting examples of suitable crosslinking agents include imidazolidinones, polycarboxylic acids and mixtures thereof. In one example, the crosslinking agent comprises a urea glyoxal adduct crosslinking agent, for example a dihydroxyimidazolidinone, such as dihydroxyethylene urea (“DHEU”). A crosslinking agent can be present in the filament-forming composition and/or filament of the present invention to control the filament's solubility and/or dissolution in a solvent, such as a polar solvent. Use of crosslinking agents provides a means for regulating the dissolution performance of filaments of the present invention.

In another example, an additive comprises a rheology modifier, such as a shear modifier and/or an extensional modifier. Non-limiting examples of rheology modifiers include but are not limited to polyacrylamide, polyethylene oxides, polyurethanes and polyacrylates that may be used in the filaments of the present invention. Non-limiting examples of rheology modifiers are commercially available from The Dow Chemical Company (Midland, Mich.).

In yet another example, an additive comprises one or more colors and/or dyes that are incorporated into the filaments of the present invention to provide a visual signal when the filaments are exposed to conditions of intended use and/or when a microorganism is released from the filaments when the filament dissolves and/or when a filament's morphology changes.

In yet another example, an additive comprises one of more sensorial agents that are incorporated into the filaments of the present invention to provide cooling, warming or other sensorial signals during use. Non-limiting examples of sensorial agents include cooling sensates and/or warming sensates, perfumes, odor control agents, and mixtures thereof.

In still yet another example, an additive comprises one or more release agents and/or lubricants. Non-limiting examples of suitable release agents and/or lubricants include fatty acids, fatty acid salts, fatty alcohols, fatty esters, sulfonated fatty acid esters, fatty amine acetates, fatty amide, silicones, aminosilicones, fluoropolymers, and mixtures thereof. In one example, the release agents and/or lubricants are applied to the filament, in other words, after the filament is formed. In one example, one or more release agents/lubricants are applied to the filament prior to collecting the filaments on a collection device to form a nonwoven. In another example, one or more release agents/lubricants are applied to a nonwoven web formed from the filaments of the present invention prior to contacting one or more nonwoven webs, such as in a stack of nonwoven webs. In yet another example, one or more release agents/lubricants are applied to the filament of the present invention and/or nonwoven comprising the filament prior to the filament and/or nonwoven contacting a surface, such as a surface of equipment used in a processing system so as to facilitate removal of the filament and/or nonwoven web and/or to avoid layers of filaments and/or nonwoven webs of the present invention sticking to one another, even inadvertently. In one example, the release agents/lubricants comprise particulates.

In even still yet another example, an additive comprises one or more anti-blocking and/or detackifying agents. Non-limiting examples of suitable anti-blocking and/or detackifying agents include starches, starch derivatives, crosslinked polyvinylpyrrolidone, crosslinked cellulose, microcrystalline cellulose, silica, metallic oxides, calcium carbonate, talc, mica, and mixtures thereof.

In one example, an additive comprises a pH buffering agent, for example sodium citrate dehydrate.

“Conditions of intended use” as used herein means the temperature, moisture, such as water, bodily fluids, such as saliva and/or menstrual fluids, pH, and/or mechanical conditions that a filament of the present invention is exposed to when the filament is used for one or more of its designed purposes. For example, if a filament is designed to be used in a disposable absorbent article, such as a feminine hygiene product, such as tampons, feminine hygiene pads, sanitary napkins, pantiliners, wipes, baby care products, such as diapers, baby pants, toddler pants, overnight pants, swim pants, and/or wipes, adult incontinence products, such as adult incontinence pads and/or adult incontinence pants, paper products, such as bath tissue, facial tissue, paper towels, and wipes, skin care products, such as skin care wipes, beauty care products, such as beauty care wipes, hair care products, such as hair care wipes, and/or pet care products such as pet care wipes, the conditions of intended use will include those temperature, moisture, urine, sweat, menstrual or other bodily fluids, pH, and/or mechanical, such as friction, conditions present during use of the products. In another example, if a filament is designed to be used in an oral care product, the conditions of intended use will include those temperature, moisture, saliva, pH, and/or mechanical conditions present during use of the oral care product by a human or in an animal. Likewise, if a filament is designed to be used in a household cleaning product, the conditions of intended use will include the temperature, moisture, such as resulting from wetting the filament with water, and/or mechanical conditions present during use of the household cleaning product. In addition to the above, the filaments of the present invention may be used in food for humans and/or pets. “Treats” as used herein with respect to treating a surface or environment means that one or more of the microorganisms provide a benefit to a surface or environment. Treats includes regulating and/or immediately improving a surface's or environment's appearance, cleanliness, smell, purity and/or feel. In one example treating in reference to treating a keratinous tissue (for example skin and/or hair) surface means regulating and/or immediately improving the keratinous tissue's cosmetic appearance and/or feel. For instance, “regulating skin, hair, or nail (keratinous tissue) condition” includes: thickening of skin, hair, or nails (e.g, building the epidermis and/or dermis and/or sub-dermal [e.g., subcutaneous fat or muscle] layers of the skin, and where applicable the keratinous layers of the nail and hair shaft) to reduce skin, hair, or nail atrophy, increasing the convolution of the dermal-epidermal border (also known as the rete ridges), preventing loss of skin or hair elasticity (loss, damage and/or inactivation of functional skin elastin) such as elastosis, sagging, loss of skin or hair recoil from deformation; melanin or non-melanin change in coloration to the skin, hair, or nails such as under eye circles, blotching (e.g., uneven red coloration due to, e.g., rosacea) (hereinafter referred to as “red blotchiness”), sallowness (pale color), discoloration caused by telangiectasia or spider vessels, and graying hair.

In one example, one or more of the microorganisms may perform its function (i.e., treat) upon use and/or consumption by an animal, for example a mammal, such as a human, by way of mouth, nose, eyes, ears, skin pores, rectum, vagina, or other orifice or wound (such as delivering a microorganism by wound dressing) in the animal. Non-limiting examples of products comprising filaments of the present invention that are intended for such uses and/or consumption include feminine hygiene products (for example tampons, pads, panty liners), baby care products, oral care products such as teeth whitening products, gum health products, floss, medicinal products, dietary products (for example delivered in a new food form), personal health care, beauty care and pet care products, and mixtures thereof.

“Weight ratio” as used herein means the weight of filament-forming material (g or %) on a dry weight basis in the filament to the weight of microorganism(s) (g or %) on a dry weight basis in the filament.

“Hydroxyl polymer” as used herein includes any hydroxyl-containing polymer that can be incorporated into a filament of the present invention, for example as a filament-forming material. In one example, the hydroxyl polymer of the present invention includes greater than 20% and/or greater than 50% and/or greater than 90% by weight hydroxyl moieties.

“Non-cellulose-containing” as used herein with respect to the filaments of the present invention means that less than 5% and/or less than 3% and/or less than 1% and/or less than 0.1% and/or 0% by weight of cellulose polymer, cellulose derivative polymer and/or cellulose copolymer is present in filament. In one example, “non-cellulose-containing” means that less than 5% and/or less than 3% and/or less than 1% and/or less than 0.1% and/or 0% by weight of cellulose polymer is present in filament.

“Polar solvent-soluble material” as used herein with respect to the filaments of the present invention means a material that is miscible in a polar solvent. In one example, a polar solvent-soluble material is miscible in alcohol and/or water. In other words, a polar solvent-soluble material is a material that is capable of forming a stable (does not phase separate for greater than 5 minutes after forming the homogeneous solution) homogeneous solution with a polar solvent, such as alcohol and/or water at ambient conditions.

“Alcohol-soluble material” as used herein with respect to the filaments of the present invention means a material that is miscible in alcohol. In other words, a material that is capable of forming a stable (does not phase separate for greater than 5 minutes after forming the homogeneous solution) homogeneous solution with an alcohol at ambient conditions. “Water-soluble material” as used herein with respect to the filaments of the present invention means a material that is miscible in water. In other words, a material that is capable of forming a stable (does not separate for greater than 5 minutes after forming the homogeneous solution) homogeneous solution with water at ambient conditions.

“Ambient conditions” as used herein means 23° C.±2.2° C. and a relative humidity of 50%±10%.

“Length” as used herein, with respect to a filament, means the length along the longest axis of the filament from one terminus to the other terminus. If a filament has a kink, curl or curves in it, then the length is the length along the entire path of the filament.

“Diameter” as used herein, with respect to a filament, is measured according to the Diameter Test Method described herein. In one example, a filament of the present invention exhibits a diameter of less than 100 μm and/or less than 75 μm and/or less than 50 μm and/or less than 25 μm and/or less than 20 μm and/or less than 15 μm and/or less than 10 μm and/or greater than 1 μm and/or greater than 3 μm and/or greater than 5 μm and/or greater than 7 μm.

“Triggering condition” as used herein in one example means anything, such as an act or event, that serves as a stimulus and initiates a change in the filament, such as a loss or altering of the filament's physical structure, swelling, gelling or dissolution of the filament and/or a release of a microorganism from the filament. In another example, the triggering condition may be present in an environment, such as water, when a filament of the present invention is added to the water. In other words, nothing changes in the water except for the fact that the filament of the present invention is added to the water.

“Morphology changes” as used herein with respect to a filament's morphology changing means that the filament experiences a change in its physical structure. Non-limiting examples of morphology changes for a filament of the present invention include dissolution, melting, swelling, shrinking, breaking into pieces, exploding, lengthening, shortening, and combinations thereof. The filaments of the present invention may completely or substantially lose their filament physical structure or they may have their morphology changed or they may retain or substantially retain their filament physical structure as they are exposed to conditions of intended use.

“By weight on a dry filament basis” means the weight of the filament measured immediately after removing the filament from a dessicator with a dessicant, for example a dessicator/dessicant commercially available from Desican Inc. under the tradename M-3003-66 which uses a molecular sieve dessicant, and in which the filament has equilibrated in the dessicator for at least 24 hours prior to removing from the dessicator. The weight of the filament is measured in a conditioned room at a temperature of 23° C.±2.2° C. and a relative humidity of 50%±10% immediately after removing the filament from the dessicator/dessicant. In one example, “by weight on a dry filament basis” means that the filament may comprise less than 20% and/or less than 15% and/or less than 10% and/or less than 7% and/or less than 5% and/or less than 3%, but greater than 0% by weight on a dry filament basis of moisture, such as water, for example free water.

“Total level” as used herein, for example with respect to the total level of one or more additives and/or microorganisms, present in the filament, means the sum of the weights, the sum of the colony forming units/g of filament (“CFU/g”) of microorganisms, or weight percent of all of the other (non-microorganism) additives. In other words, a filament may comprise at least 10³ CFU/g and/or at least 10⁴ CFU/g and/or at least 10⁵ CFU/g and/or at least 10⁶ CFU/g and/or at least 10⁷ CFU/g and/or at least 10⁸ CFU/g by weight on a dry filament basis of one or more microorganisms. In another example, a filament of the present invention may comprise one or more non-microorganism additives in the filament at a total level of at least 1% and/or at least 5% and/or at least 10% and/or at least 20% and/or up to 50% by weight on a dry filament basis.

“Labile microorganism” as used herein means a microorganism that is likely to undergo change, for example a microorganism that is likely to lose all or a substantial part (at least a 1 log viability loss or greater as measured according to the Viability/Count Test Method described herein) when exposed to stresses, for example humidity, temperature, shear, aerobic conditions. A non-limiting example of a stress is exposing the microorganism to 30° C./30% RH conditions for 28 and/or 56 days. The L. fermentum 297R1 in Example 1 and B. infantis 35624 in Example 2 below are examples of labile microorganisms as used herein.

As used herein, the articles “a” and “an” when used herein, for example, “an anionic surfactant” or “a fiber” is understood to mean one or more of the material that is claimed or described.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

### Filament

The filaments of the present invention comprise a filament-forming material and one or more microorganisms. In one example, the one or more microorganisms are present within the filament rather than being present only as a surface coating or partially embedded on the filaments. The filaments of the present invention may exhibit an average fiber diameter of greater than 1 μm. In one example, the filaments of the present invention exhibit an average fiber diameter of less than 50 μm and/or less than 25 μm and/or less than 10 μm but greater than 1 μm as measured according to the Diameter Test Method described herein.

In one example, at least one of the microorganisms present in a filament of the present invention exhibits less than a 2.7 log and/or less than a 2.5 log and/or less than a 2.25 log less than a 2 log and/or less than a 1 log viability loss after being exposed to 30° C./30% RH conditions for 28 days as measured according to the Viability/Count Test Method described herein.

In another example, at least one of the microorganisms present in a filament of the present invention exhibits less than a 6.5 log and/or less than a 6 log and/or less than a 5 log and/or less than a 4 long and/or less than a 3 log and/or less than a 2.75 log and/or less than a 2.5 log and/or less than a 2.25 log viability loss after being exposed to 30° C./30% RH conditions for 56 days as measured according to the Viability/Count Test Method described herein.

In one example, at least one of the microorganisms present in the filament of the present invention is releasable from the filament when exposed to conditions of intended use.

In one example, the total level of filament-forming materials present in a filament of the present invention is less than 90% and/or less than 80% and/or less than 70% and/or less than 60% by weight on a dry filament basis and the total level of the one or more microorganisms present in the filament is less than 50% and/or greater than 1% by weight on a dry filament basis.

In addition to the filament-forming materials and the microorganisms, the filaments of the present invention may comprise one or more stabilizing agents. In one example, the total level of stabilizing agents present in the filament of the present invention is less than 60% and/or greater than 10% by weight on a dry filament basis.

In another example, the filament may further comprise an antioxidant. The total level of antioxidants present in a filament of the present invention is less than 1% and/or to 0% by weight on a dry filament basis.

In still another example, the filament of the present invention comprises from about 20% and/or from about 30% and/or from about 40% to about 50% and/or to about 60% and/or to about 70% by weight on a dry filament basis of a filament-forming material, such as polyvinyl alcohol polymer and/or a starch polymer, and at least 10³ CFU/g and/or at least 10⁴ CFU/g and/or at least 10⁵ CFU/g and/or at least 10⁶ CFU/g and/or at least 10⁷ CFU/g and/or at least 10⁸ CFU/g by weight on a dry filament of one or more microorganisms after being exposed to 30° C./30% RH conditions for 28 and/or 56 days as measured according to the Viability/Count Test Method described herein. In one example, the filaments of the present invention may be meltblown filaments. In another example, the filaments of the present invention may be spunbond filaments. In another example, the filaments may be hollow filaments prior to and/or after release of one or more of its microorgansims.

The filaments of the present invention may be hydrophilic or hydrophobic. The filaments may be surface treated and/or internally treated to change the inherent hydrophilic or hydrophobic properties of the filament.

In one example, the filament exhibits an average diameter of less than 100 μm and/or less than 75 μm and/or less than 50 μm and/or less than 25 μm and/or less than 20 μm and/or less than 15 μm and/or less than 10 μm and/or greater than 1 μm and/or greater than 3 μm and/or greater than 5 μm and/or greater than 7 μm as measured according to the Diameter Test Method described herein. In another example, the filament of the present invention exhibits a diameter of greater than 1 μm as measured according to the Diameter Test Method described herein. The diameter of a filament of the present invention may be used to control the rate of release of one or more microorganisms present in the filament and/or the rate of loss and/or altering of the filament's physical structure.

In one example, one or more microorganisms are released from the filament of the present invention upon dissolution of the filament as measured according to the Dissolution Test Method described herein. In another example, one or more microorganisms are released from the filament of the present invention upon dissolution of the filament in less than 60 minutes and/or less than 45 minutes and/or less than 30 minutes and/or less than 15 minutes and/or less than 10 minutes and/or less than 5 minutes and/or less than 1 minute as measured according to the Dissolution Test Method described herein. In another example, one or more microorganisms are released from the filament of the present invention upon dissolution of the filament when the filament is exposed to conditions of intended use.

The filament may comprise two or more different microorganisms. In one example, the filament comprises two or more different microorganisms, wherein the two or more different microorganisms are compatible with one another. In another example, the filament comprises two or more different microorganisms, wherein the two or more different microorganisms are incompatible with one another.

In one example, the filament may comprise a microorganism within the filament and a microorganism on an external surface of the filament, such as a surface coating or partially embedded in the filament. The microorganism on the external surface of the filament may be the same or different from the microorganism present in the filament. If different, the microorganisms may be compatible or incompatible with one another.

In one example, one or more microorganisms may be uniformly distributed or substantially uniformly distributed throughout the filament. In another example, one or more microorganisms may be distributed as discrete regions within the filament such that one portion of the filament contains microorganisms and another portion of the filament is void of microorganisms. In still another example, at least one microorganism is distributed uniformly or substantially uniformly throughout the filament and at least another microorganism is distributed as one or more discrete regions within the filament. In still yet another example, at least one microorganism is distributed as one or more discrete regions within the filament and at least another microorganism is distributed as one or more discrete regions different from the first discrete regions within the filament. In even another example, the filament of the present invention may contain one or more microorganisms such that a cross-section of the filament comprises at least two microorganisms. Still yet another example of the filament of the present invention is a bicomponent filament wherein the core contains one or more microorganisms and the sheath is void of microorganisms or the core is void of microorganisms and the sheath contains microorganisms or the core contains a first microorganism and the sheath contains a second micoorgansim different from the first microorganism or the core contains one or more microorganisms and the sheath contains one or more filament-forming materials. In another example of a bicomponents filament, such as a side-by-side bicomponent filament, one side may contain a microorganism and the other side may be void of microorganisms or one side may contain a first microorganism and the other side may contain a second microorganism different from the first microorganism.

The filaments may be used as discrete articles. In one example, the filaments may be applied to and/or deposited on a carrier substrate, which may then be used in or as a wipe, paper towel, bath tissue, facial tissue, disposable absorbent article, such as a sanitary napkin, a tampon, a feminine hygiene pad, a pantiliner, a diaper, baby pants, toddler pants, overnight pants, swim pants, an adult incontinence article, such as adult incontinence pads and/or adult incontinence pants, and the like, washcloth, dryer sheet, laundry sheet, laundry bar, dry cleaning sheet, netting, filter paper, fabric, clothing, undergarment, and the like. In another example, the filament and/or portions (fragments) of the filament may be incorporated into an article, such as a tablet and/or capsule and/or kibble.

In one example, the filaments of the present invention are water-soluble.

In another example, the filaments of the present invention edible, ingestible, and/or consumable by humans and/or animals. In other words, the filaments of the present invention are made from suitable materials, for example filament-forming materials and microorganisms, that are safe for human and/or animal ingestion and/or consumption.

### Filament-forming Materials

The filaments of the present invention comprise one or more filament-forming materials. The filament-forming materials may be present in the filament at a total level of from about 10% to about 90% and/or from about 20% to about 80% and/or from about 30% to about 70% and/or from about 40% to about 60% by weight on a dry filament basis.

In one example, the filament-forming material may comprise a polar solvent-soluble material, such as an alcohol-soluble material and/or a water-soluble material. Non-limiting examples of polar solvent-soluble materials include polar solvent-soluble polymers. The polar solvent-soluble polymers may be synthetic or of natural original and may be chemically and/or physically modified. In one example, the polar solvent-soluble polymers exhibit a weight average molecular weight of at least 10,000 g/mol and/or at least 20,000 g/mol and/or at least 40,000 g/mol and/or at least 80,000 g/mol and/or at least 100,000 g/mol and/or at least 1,000,000 g/mol and/or at least 3,000,000 g/mol and/or at least 10,000,000 g/mol and/or at least 20,000,000 g/mol and/or to about 40,000,000 g/mol and/or to about 30,000,000 g/mol.

In one example, the water-soluble hydroxyl polymer is selected from the group consisting of: polyvinyl alcohols, hydroxymethylcelluloses, hydroxyethylcelluloses, hydroxypropylmethylcelluloses and mixtures thereof. A non-limiting example of a suitable polyvinyl alcohol includes those commercially available from Sekisui Specialty Chemicals America, LLC (Dallas, Tex.) under the CELVOL® trade name. A non-limiting example of a suitable hydroxypropylmethylcellulose includes those commercially available from the Dow Chemical Company (Midland, Mich.) under the METHOCEL® trade name including combinations with above mentioned hydroxypropylmethylcelluloses.

In one example the polyvinyl alcohols herein can be grafted with other monomers to modify its properties. A wide range of monomers has been successfully grafted to polyvinyl alcohol. Non-limiting examples of such monomers include vinyl acetate, styrene, acrylamide, acrylic acid, 2-hydroxyethyl methacrylate, acrylonitrile, 1,3-butadiene, methyl methacrylate, methacrylic acid, maleic acid, itaconic acid, sodium vinylsulfonate, sodium allylsulfonate, sodium methylallyl sulfonate, sodium phenylallylether sulfonate, sodium phenylmethallylether sulfonate, 2-acrylamido-methyl propane sulfonic acid (AMPs), vinylidene chloride, vinyl chloride, vinyl amine and a variety of acrylate esters.

In yet another example, the filament-forming material may be a film-forming material. In still yet another example, the filament-forming material may be synthetic or of natural origin and it may be chemically, enzymatically, and/or physically modified.

In even another example of the present invention, the filament-forming material may comprise a polymer selected from the group consisting of: polymers derived from acrylic monomers such as the ethylenically unsaturated carboxylic monomers and ethylenically unsaturated monomers, polyvinyl alcohol, polyacrylates, polymethacrylates, copolymers of acrylic acid and methyl acrylate, polyvinylpyrrolidones, polyalkylene oxides, starch and starch derivatives, pullulan, gelatin, hydroxypropylmethylcelluloses, methycelluloses, and carboxymethycelluloses.

In still another example, the filament-forming material may comprises a polymer selected from the group consisting of: polyvinyl alcohol, polyvinyl alcohol derivatives, starch, starch derivatives, cellulose derivatives, hemicellulose, hemicellulose derivatives, proteins, sodium alginate, hydroxypropyl methylcellulose, chitosan, chitosan derivatives, polyethylene glycol, polyethylene oxide, polyacrylamide, tetramethylene ether glycol, polyvinyl pyrrolidone, hydroxymethyl cellulose, hydroxyethyl cellulose, and mixtures thereof.

In another example, the filament-forming material comprises a polymer selected from the group consisting of: pullulan, hydroxypropylmethyl cellulose, hydroxyethyl cellulose, hydroxypropyl cellulose, polyvinyl pyrrolidone, carboxymethyl cellulose, sodium alginate, xanthan gum, tragacanth gum, guar gum, acacia gum, Arabic gum, polyacrylic acid, methylmethacrylate copolymer, carboxyvinyl polymer, dextrin, pectin, chitin, levan, elsinan, collagen, gelatin, zein, gluten, soy protein, casein, polyvinyl alcohol, starch, starch derivatives, hemicellulose, hemicellulose derivatives, proteins, chitosan, chitosan derivatives, polyethylene glycol, tetramethylene ether glycol, hydroxymethyl cellulose, and mixtures thereof.

In another example, the filament-forming material is selected from the group consisting of: polyvinyl alcohol, polyvinyl alcohol derivatives, polyethylene oxide, starch, starch derivatives, cellulose, cellulose derivatives, carboxymethyl cellulose, hydroxypropylmethyl cellulose, hydroxyethyl cellulose, hydroxypropyl cellulose, polyvinyl pyrrolidone, sodium alginate, xanthan gum, tragacanth gum, guar gum, acacia gum, Arabic gum, polyacrylic acid, methylmethacrylate copolymer, carboxyvinyl polymer, chitosan, chitosan derivatives, polyethylene glycol, hemicellulose, hemicelluloses derivatives, polyacrylamide, and copolymers and mixtures thereof.

In one example, the polar solvent-soluble polymers are selected from the group consisting of: alcohol-soluble polymers, water-soluble polymers and mixtures thereof. Non-limiting examples of water-soluble polymers include water-soluble hydroxyl polymers, water-soluble thermoplastic polymers, water-soluble biodegradable polymers, water-soluble non-biodegradable polymers and mixtures thereof. In one example, the water-soluble polymer comprises polyvinyl alcohol. In another example, the water-soluble polymer comprises carboxymethylcellulose. In another example, the water-soluble polymer comprises starch. In yet another example, the water-soluble polymer comprises polyvinyl alcohol and starch.

### Microorganisms

The filaments of the present invention comprise one or more microorganisms. The microorganisms may be selected from the group consisting of: prokaryotes, eukaryotes, viruses, bacteriophages, and mixtures thereof. Non-limiting examples of prokaryotes include bacteria and archaea. Non-limiting examples of eukaryotes include fungi.

### Bacteria

Bacteria suitable for use in the present invention include gram-positive cocci, gram-positive bacilli and gram-negative baccili. Non-limiting example of bacteria for use in the filaments of the present invention include microbes isolated from human and animal microbiota (the aggregate of microorganisms that reside on the surface and in deep layers of skin, in the saliva, in the oral mucosa, in the vaginal mucosa, in the conjunctiva, and in the gastrointestinal tracts.

In one example, the bacteria is a probiotic.

A probiotic is a bacteria that provides a beneficial health and/or welfare effect on its host, such as humans and/or animals. Non-limiting examples of probiotics for use in the filaments of the present invention include Bifidobacteria species, Lactobacillus species, Lactococcus species, Pediococcus species, Leuconostoc species, Sporolactobacillus species, and Bacillus species and mixtures thereof.

Non-limiting examples of Bifidobacteria species include Bifidobacterium adolescentis, Bifidobacterium bifidum, Bifidobacterium animalis, Bifidobacterium thermophilum, Bifidobacterium breve, Bifidobacterium ion gum, Bifidobacterium infantis and Bifidobacterium lactis. Specific strains of Bifidobacteria useful as probiotics include Bifidobacterium breve strain Yakult, Bifidobacterium breve R070, Bifidobacterium lactis Bb12, Bifidobacterium longum R023, Bifidobacterium bifidum R071, Bifidobacterium infantis 35624, Bifidobacterium infantis R033, Bifidobacterium longum BB536, Bifidobacterium animalis AHC7, and Bifidobacterium longum SBT-2928.

Non-limiting examples of Lactobacillus species for use in the filaments of the present invention include Lactobacillus sporogenes, Lactobacillus jensenii, Lactobacillus vaginalis, Lactobacillus gallinarum, Lactobacillus coleohominis, and Lactobacillus iners, Lactobacillus bulgaricus, Lactobacillus cereale, Lactobacillus delbrukeii, Lactobacillus rhamnosus, Lactobacillus thermophilus, Lactobacillus paracasai sp. paracasai, Lactobacillus helveticus, Lactobacillus acidophilus, Lactobacillus brevis, Lactobacillus ulgaricus, Lactobacillus casei, Lactobacillus cellobiosus, Lactobacillus crispatus, Lactobacillus curvatus, Lactobacillus fermentum, Lactobacillus GG (Lactobacillus rhamnosus or Lactobacillus casei subspecies rhamnosus), Lactobacillus gasseri, Lactobacillus johnsonii, Lactobacillus plantarum, Lactobacillus reuteri, Lactobacillus salivarius, and mixtures thereof. Lactobacillus plantarum 299v strain originates from sour dough. Lactobacillus plantarum itself is of human origin. Other probiotic strains of Lactobacillus are Lactobacillus acidophilus BG2FO4, Lactobacillus acidophilus INT-9, Lactobacillus plantarum ST3 1, Lactobacillus reuteri, Lactobacillus johnsonii LA1, Lactobacillus acidophilus NCFB 1748, Lactobacillus casei Ski rota, Lactobacillus acidophilus NCFM, Lactobacillus acidophilus DDS-1, Lactobacillus delbrueckii subspecies delbrueckii, Lactobacillus delbrueckii subspecies bulgaricus type 2038, Lactobacillus acidophilus SBT-2062, Lactobacillus brevis, Lactobacillus salivarius UCC 118, Lactobacillus fermentum 297R1, Lactobacillus reuteri Grant L1, Lactobacillus crispatus 330L1, Lactobacillus and Lactobacillus paracasei subsp paracasei F 19. In one example the microorganism comprises a species of Lactobacillus include L. caesi, L. acidophilus, L. plantarum, and L. rhamnosus.

A non-limiting examples of Lactococcus species for use in the filaments of the present invention includes Lactococcus lactis.

Non-limiting examples of Pediococcus species for use in the filaments of the present invention include Pediococcus acidilactici, Pedioccocus pentosaceus, Pedioccocus urinae, and mixtures thereof.

A non-limiting example of a Leuconostoc species for use in the filaments of the present invention includes Leuconostoc mesenteroides.

A non-limiting example of a Sporolactobacillus species for use in the filaments of the present invention includes Sporolactobacillus inulinus.

Non-limiting examples of Bacillus species for use in the filaments of the present invention include Bacillus coagulans, Bacillus subtilis, Bacillus laterosporus, Bacillus laevolacticus, and mixtures thereof.

Other probiotic microbes that may be present in the filaments of the present invention include the gram-positive facultative anaerobe Streptococcus thermophilus, Enterococcus faecium SF68.

In one example, the probiotic is Bifantis™ 35624 (bifido bactierium, Chr. Hansen, Denmark) and/or Bifidobacterium infantis 35624. Non-limiting examples of other suitable probiotics include probiotics from strains of Bifidobacterium isolated from resected and washed human gastrointestinal tract. An example includes Bifidobacterium infantis strain designated UCC35624, described as being deposited at the National Collections of Industrial and Marine Bacteria Ltd (NCIMB) on Jan. 13, 1999, and accorded the accession number NCIMB 41003 and described in U.S. Pat. No. 7,195,906. Suitable examples of probiotics useful herein comprise strains of Bifidobacterium longum infantis (NCIMB 35624), Lactobacillus johnsonii (CNCM 1-1225), Bifidobacterium lactis (DSM20215), Lactobacillus paracasei (CNCM 1-2216), and mixtures thereof. Further non-limiting examples of probiotics useful herein are described in WO 03/010297 A1, WO 03/010298 A1, WO 03/010299 A1 (all published Feb. 6, 2003 and assigned to Alimentary Health Ltd) and US Patent Application Publication No. US2012/0276143.

In one example, the probiotic comprises Bifidobacterium strain AH1714 and/or Bifidobacterium longum strain UCC35624. A deposit of Bifidobacterium longum strain AH1714 was made at the National Collections of Industrial and Marine Bacteria Limited (NCIMB) Ferguson Building, Craibstone Estate, Bucksbum, Aberdeen, AB21 9YA, Scotland, UK on Nov. 5, 2009 and accorded the accession number NCIMB 41676. A deposit of Bifidobacterium longum strain UCC35624 was made at the National Collections of Industrial and Marine Bacteria Limited (NCIMB) Ferguson Building, Craibstone Estate, Bucksburn, Aberdeen, AB21 9YA, Scotland, UK on Jan. 13, 1999 and accorded the accession number NCIMB 41003. The Bifidobacterium longum strain may be a genetically modified mutant or it may be a naturally occurring variant thereof. In one example, the Bifidobacterium longum strain is in the form of viable cells. In another example, the Bifidobacterium longum strain is in the form of non-viable cells

In another example, the probiotic comprises Bifidobacterium strain AH121A. A deposit of Bifidobacterium longum strain AH121A was made at the National Collections of Industrial and Marine Bacteria Limited (NCIMB) Ferguson Building, Craibstone Estate, Bucksburn, Aberdeen, AB21 9YA, Scotland, UK on Nov. 5, 2009 and accorded the accession number NCIMB 41675. Non-limiting examples of microorganisms can include strains of Streptococcus lactis, Streptococcus cremoris, Streptococcus diacetylactis, Streptococcus thermophilus, Lactobacillus bulgaricus, Lactobacillus acidophilus (e.g., Lactobacillus acidophilus strain), Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus reuteri, Bifidobacterium longum, Bifidobacterium infantis, Bifidobacterium bifidum, Bifidobacterium animalis, Bifidobacterium pseudolongum, Saccharomyces boulardii, Pediococcus cerevisiae, Lactobacillus salivarius, Bacillus coagulans, and combinations thereof. Such probiotics, in one example, can be present in the filaments of the present invention at from about 0.025% to about 10% and/or from about 0.025% to about 5% and/or from about 0.025% to about 3% and/or from about 0.025% to about 1%, by weight on a dry filament basis.

### Fungi

Non-limiting examples of suitable fungi for use in the filaments of the present invention include Penicillum, Saccharomyces cerevisiae, and mixtures thereof.

### Viruses

Suitable viruses for use in the filaments of the present invention include all 7 groups of viruses. These include Group I: double-stranded DNA viruses; Group II: single-stranded DNA viruses; Group III: double-stranded RNA viruses; Group IV: positive-sense single-stranded RNA viruses; Group V: negative-sense single-stranded RNA viruses; Group VI: reverse transcribing RNA viruses and Group VII: reverse transcribing DNA viruses. Non-limiting examples include vaccines for human and animals.

### Bacteriophages

Suitable bacteriophages for use in the filaments of the present invention include Salmonella, E. coli, Pseudomonas, Bacillus, Listeria, Burkholderia, S. aureus, and S. mutans phages.

### Prebiotics

In addition to one or more microorganisms, the filaments of the present invention may further comprise one or more prebiotics.

Non-limiting examples of suitable prebiotics include inulin, lactose, raffinose, stachyose, fructo-oligossacharides, gluco-oligosaccharides, lactoferrin, mannan oligosaccharides, glucan oligosaccharides, isomalto-oligo saccharides, lactosucrose, polydextrose, soybean oligosaccharides, xylo-oligosaccharides, and mixtures thereof.

Other non-limiting examples of suitable prebiotics include human milk oligosaccharides as disclosed in US2013281948 A1, for example lactose, 2′-fucosyllactose, 3′-fucosyllactose, difucosyllactose, lacto-N-tetraose (type 1), lacto-N-neo-tetraose (type 2), lacto-N-fucopentaoses I, II, III, IV and V, lacto-N-fucohexaose I, lacto-N-hexaose, lacto-N-neohexaose, fucosyllacto-Nhexaose I and IV, fucosyllacto-N-neohexaose, lacto-N-difuco-hexaoses I and II, lacto-Noctaoses, sialya2-3lactose, sialya2-6lactose, sialyl-lacto-N-tetraose a, b and c, and disialyl-lacto-N-tetraose, and mixtures thereof.

Still other non-limiting examples of suitable prebiotics include fucose-a(1®2)galalactose b as a disaccharide unit, 2′-fucosyllactose, 3′-fucosyllactose, lacto-N-difuco-tetraose, lacto-N-difuco-hexose I, lacto-N-difuco-hexose II, lacto-N-fucopentaose I, lacto-N-fucopentaose II, lacto-N-fucopentaose III, lacto-N-fucopentaose V, and mixtures thereof.

In one example, the prebiotics may comprise carob bean, citrus pectin, rice bran, locust bean, fructooligosaccharide, oligofructose, galactooligosaccharide, citrus pulp, annanoligosaccharides, arabinogalactan, lactosucrose, glucomannan, polydextrose, apple pomace, tomato pomace, carrot pomace, cassia gum, gum karaya, gum talha, gum arabic, and combinations thereof. Such prebiotics, in one example, may be present in the filaments of the present invention at from about 1% to about 85% and/or from about 10% to about 60% and/or from about 20% to about 50% by weight on a dry filament basis.

### Stabilizing Agents

The filaments of the present invention may comprise one or more stabilizing agents. When present in the filament, the stabilizing agents may be present in the filament at a level of from about 0% to about 60% and/or from about 10% to about 50% by weight on a dry filament basis.

The stabilizing agent may comprise a carbohydrate and/or a protein. The carbohydrate may be present in the filaments at a level of from about 0% to about 50% and/or from about 10% to about 40% by weight on a dry filament basis. The carbohydrate may be selected from the group consisting of: monosaccharides, disaccharides, oligosaccharides, polysaccharides, and mixtures thereof. Non-limiting examples of suitable carbohydrates include sucrose, trehalose, glycerol, glucose, mannitol, sorbitol, adonitol, betaine (N,N,N-trimethylglycine), lactose, fructo-oligosaccharides (FOS), polyfructoses, for example, inulin, pectin, 6-glucans, resistant starches, for example high amylose starch, dextrans, acacia gum, guar and locust bean gum, agar, carrageenans, xanthan and maltodextrins, and mixtures thereof.

The protein may be present in the filaments at a level of from about 0% to about 30% and/or from about 1% to about 20% by weight on a dry filament basis. The protein may be selected from the group consisting of albumen, arginine/lysine polypeptide, collagen and hydrolyzed collagen, gelatin and hydrolyzed gelatin, glycoproteins, milk protein, casein, for example sodium caseinate, whey protein, soy protein, barley protein, serum albumin, meat, fish, seafood, poultry, egg proteins, silk, soybean, corn, peanut, cottonseed, sunflower, pea, wheat protein, wheat germ protein, gluten-protein, zein and any isolate or hydrolyzed of any vegetable protein, such as soy protein isolate and/or hydrosylate, barley protein isolate and/or hydrosylate, and mixtures thereof.

### Antioxidants

The filaments of the present invention may comprise one or more antioxidants. When present, the antioxidants may be present in the filament at a level of from about 0.01% to about 1% and/or from about 0.1% to about 0.5% and/or from about 0.1% to about 0.2% by weight on a dry filament basis.

Non-limiting examples of antioxidants that may be present in the filaments of the present invention include the following.

Rice bran derivatives have been shown to have more than a hundred (100) potent antioxidants including vitamin E and its isomers (tocopherols (T) and tocotrienols (T3)), collectively referred to as tocols. A tocol-rich substance is a mixture containing one or more compounds selected from tocopherols (T), tocotrienols, and tocotrienollike (T3-like) compounds. Stabilized rice bran is the highest natural source of vitamin E.

Additional antioxidants in stabilized rice bran derivatives include, but are not limited to, y-oryzanol, (3-carotene, several known flavanoids, phytosterols, lipoic acid, ferulic acid and inositol hexaphospate (i.e., “IP6”). Some of these compounds are present in stabilized rice bran derivatives at concentrations which are much higher than in any of the known natural sources of the compounds. Ferulic acid, for example, is a phytochemical found in seeds of plants such as in brown rice, whole wheat and oats, as well as in coffee, apple, artichoke, peanut, orange and pineapple. Ferulic acid protects our cells from ultraviolet rays and neutralizes reactive oxygen species in the body, thereby preventing the reactive oxygen species from causing damage to our DNA. Being an antioxidant, it also reduces the level of cholesterol and triglyceride in the body and thus lowers the risk of heart diseases. IP6 is a phosphorylated form of inositol commonly found in fiber-rich plant foods. IP6 is hydrolyzed by phytase enzymes in the digestive tract to yield inositol. IP6 supports a cell's natural defense against damaging hydroxyl free radicals by chelating with reactive iron. In combination with probiotics, antioxidants provide exceptional additional defense and increase the immune system's ability to resist invasive pathogens associated with gastrointestinal disorders.

In one example, the antioxidants present in the filaments may be selected from the group consisting of: carotenoids, such as lycopene, beta-carotene, lutein, xanthophylls, vitamin A, tocopherols, vitamin C, and mixtures thereof.

In another example, the antioxidants present in the filaments may be selected from the group consisting of propyl gallate, butylated hydroxytoluene (BHT), butylated hydroxyanisole (BHA), Vitamin C, Vitamin A, Vitamin E, beta-carotene, and mixtures thereof.

### Dietary Fibers

The filaments of the present invention may further comprise dietary fibers. Non-limiting examples of dietary fibers can include, but are not limited to inulin, agar, beta-glucans, chitins, dextrins, lignin, cellulose, modified cellulose, cellulose ethers, hemicelluloses, non-starch polysaccharides, reduced starch, polycarbophil, partially hydrolyzed guar gum, wheat dextrin, and combinations thereof.

### Method for Making Filament

The filaments of the present invention are produced by spinning a filament-forming composition comprising one or more filament-forming materials and one or more microorganisms.

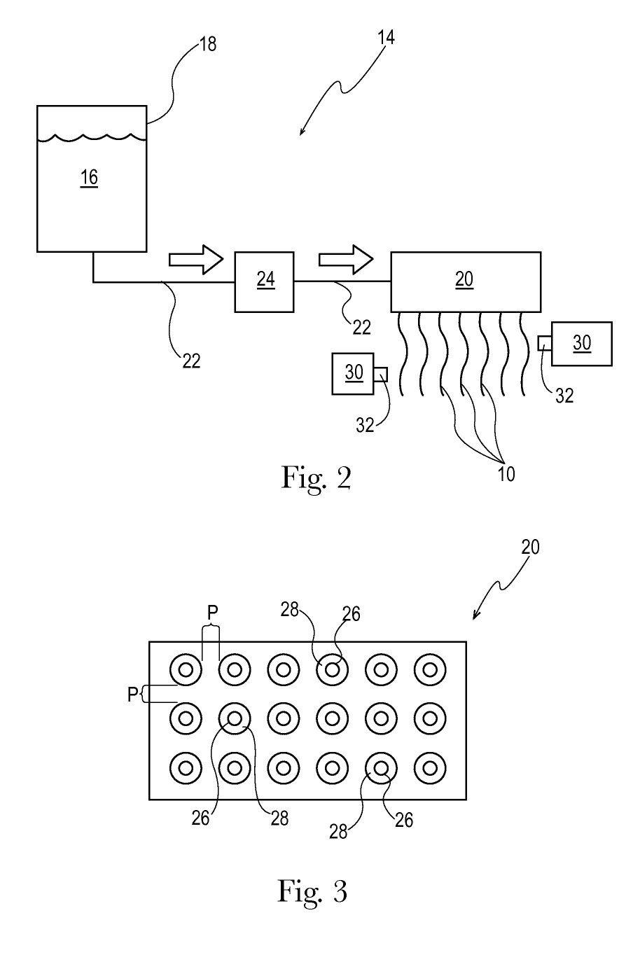

In one example, as shown in FIG. 2, a method 14 for making a filament 10 of the present invention comprises the steps of:
a. providing a filament-forming composition 16, for example a filament-forming liquid composition suitable for making filaments, comprising one or more filament-forming materials, one or more microorganisms, and one or more stabilizing agents, from a source 18, such as a tank, for example a pressurized tank suitable for batch operations; and
b. spinning the filament-forming composition 16 from a die 20, such as a meltblow die, to produce one or more filaments 10 of the present invention.

The filament-forming composition 16 may be in fluid communication with the die 20 via suitable piping 22 as shown with the arrows. A pump 24 (for example a Zenith®, type PEP II pump having a capacity of 5.0 cubic centimeters per revolution (cc/rev), manufactured by Parker Hannifin Corporation, Zenith Pumps division, of Sanford, N.C., USA) may be used to pump the filament-forming composition 16 to the die 20. The filament-forming composition's 16 flow to the die 20 may be controlled by adjusting the flowrate of the pump 20.

The die 20 as shown in FIG. 3 may comprise two or more rows of circular extrusion nozzles 26 spaced from one another at a pitch P of about 1.524 millimeters (about 0.060 inches). The nozzles 26 may have individual inner diameters of about 0.305 millimeters (about 0.012 inches) and individual outside diameters of about 0.813 millimeters (about 0.032 inches). Each individual nozzle 26 may be encircled by an annular and divergently flared orifice 28 to supply attenuation air formed by mixing steam and heated compressed air to each individual nozzle 26. The filament-forming composition 16 that is extruded through the nozzles 26 is surrounded and attenuated by generally cylindrical, attenuation air streams supplied through the orifices 28 encircling the nozzles 26 to produce the filaments 10. The filaments 10 may be dried by a drying air stream having a temperature of from about 50° C. to about 315° C. by an electrical resistance heater 30 supplied through drying ducts 32 and discharged at an angle of about 90° relative to the general orientation of the filaments 10 being spun.

During spinning of the filament-forming composition, the filament-forming composition and microorganisms contained therein are subjected to steam and attenuation air and drying air at temperatures of up to 450° C. without negatively impacting the viability of the microorganisms, for example with less than a 2 and/or less than a 1 log loss in viability. The filaments 10 may be collected on a collection device, such as a belt or fabric, in one example a belt or fabric capable of imparting a pattern, for example a non-random repeating pattern to a nonwoven web formed as a result of collecting the filaments on the belt or fabric.

In one example, the step of spinning may comprise contacting the filament with attenuation air to attenuate the filament.

The method may further comprise the step of collecting a plurality of filaments on a collection device, for example a spool or a belt or fabric, such as a patterned belt. Filaments may be collected and stored in desiccated flip top vials (commercially available from Desican Inc) and refrigerated until use.

The filaments of the present invention may exhibit an average diameter of greater than 1 μm and/or greater than 3 μm and/or greater than 5 μm and/or less than 100 μm and/or less than 70 μM.

In one example, the method of the present invention is a non-electrospinning method.

### Non-Limiting Examples

Non-limiting examples of filaments according to the present invention are produced by using the method 14 shown in FIGS. 2 and 3 as described above.

Non-limiting examples of filament-forming compositions 16 according to the present invention are shown in Table 1 below.

TABLE 1

| | Example 1 | Example 2 |
|---|---|---|
| | Level (grams) | Level (grams) |
| Ingredients of Filament | | |
| Forming Composition | | |
| Antioxidant¹ | 0.06 | 0.06 |
| Stabilizing Agent² | 4.29 | 4.29 |
| pH Buffering Agent³ | 0.15 | 0.15 |
| Protein⁴ | 1.53 | 1.53 |
| Distilled Water | 54.53 | 54.53 |
| Microorganism⁵ | 1.86 | |
| Microorganism⁶ | | 1.86 |
| Filament-forming | 10.88 | 10.88 |
| Material⁷ | | |
| Viability | | |
| Initial Probiotic | 10.37 | 10.05 |
| (Normalized at 10% | | |
| Add-on) (log CFU/g) | | |
| Unprotected Probiotic | 8.49 | 7.24 |
| After Aging 30° C./30% | | |
| RH for 28 days (log | | |
| CFU/g) | | |
| Unprotected Probiotic | — | 3.24 |
| After Aging 30° C./30% | | |
| RH for 56 days (log | | |
| CFU/g) | | |
| Probiotic in Fiber After | 8.81 | 8.14 |
| Spinning Process (log | | |
| CFU/g) | | |
| Probiotic in Fiber After | 8.24 | 7.39 |
| Aging 30° C./30% RH | | |
| for 28 days (log CFU/g) | | |
| Probiotic in Fiber After | 8 | 7.2 |
| Aging 30° C./30% RH | | |
| for 56 days (log CFU/g) | | |
| Viability Log Loss | | |
| (Initial - Condition) | | |
| Unprotected Probiotic | 1.88 | 2.81 |
| After Aging 30° C./30% | | |
| RH for 28 days (log | | |
| CFU/g) | | |
| Unprotected Probiotic | — | 6.81 |
| After Aging 30° C./30% | | |
| RH for 56 days (log | | |
| CFU/g) | | |
| Probiotic in Web After | 1.56 | 1.91 |
| Spinning Process (log | | |
| CFU/g) | | |
| Probiotic in Web After | 2.13 | 2.66 |
| Aging 30° C./30% RH | | |
| for 28 days (log CFU/g) | | |
| Probiotic in Web After | 2.37 | 2.85 |
| Aging 30° C./30% RH | | |
| for 56 days (log CFU/g) | | |
| Physical Properties of | | |
| Fibrous Elements | | |
| Dissolution of Fibrous | 12 seconds ± | 7 seconds ± |
| Element | 3 seconds | 3 seconds |
| Diameter of Fibrous | 28.1 microns ± | 33.01 microns ± |
| Element | 8.16 microns | 6.75 microns |

¹Propyl gallate (Spectrum Chemicals, Gardena, CA)
²Trehalose (Swanson Ultra, Fargo, ND)
³Sodium Citrate Dehydrate (Sigma Aldrich, St. Louis, MO)
⁴Sodium Casemate (Sigma Aldrich, St. Louis, MO)
⁵L. fermentum 297R1
⁶B infantis 35624
⁷Polyvinyl alcohol (Sekisui Specialty Chemical Company, Dallas, TX)

The filament-forming composition shown above in the non-limiting Example is prepared as follows:

1. Make up a polyvinyl alcohol solution of 23% polyvinyl alcohol by setting up a wide mouth pint jar in a water bath with over head stirrer fitted through a holed lid and a stir blade nearly as wide as the jar. Next, add 231 g distilled water. With moderate stirring, slowly add 69 g polyvinyl alcohol. Turn on water bath and heat to 70° C. When all of the polyvinyl alcohol is dissolved, turn off heat and allow to cool to 50° C. Remove from stirrer, cap, allow to sit sealed while cooling to 23° C. All of the air bubbles will be removed as it sits.

2. Make up a stock cryoprotecting solution at 25% solids.

| | | Amount (g) for a |
|---|---|---|
| Ingredients | % in Formula | 200 g batch |
| Propyl gallate | 0.23% | 0.46 |
| Trehalose | 17.77% | 35.54 |
| Sodium citrate dihydrate | 0.64% | 1.28 |
| Sodium caseinate | 6.36% | 12.72 |
| Distilled water | 75% | 150 |

Dissolve all ingredients except sodium caseinate in 75 mL distilled water by heating to 60° C. with stirring to form a trehalose solution.

Disperse the sodium caseinate in 75 mL distilled water and heat to 60° C. to form a caseinate solution. Autoclave the caseinate solution at 121° C. for 60 minutes and then cool to 23° C.

When cooled pour trehalose solution into caseinate solution. Bring weight to 200 g by rinsing trehalose solution jar. Result is 25% solids. Seal and store in refrigerator.

Next, make a filament-forming solution as follows:

1. Using a SpeedMixer or equivalent, mix 27.3 g cryoprotectant solution with 1.86 g probiotic for 4 minutes at 3500 rpm.

2. Again, using a SpeedMixer or equivalent, add 26 g of the resulting mixture from step 1 with 47.3 g of the polyvinyl alcohol solution from above by mixing 4 min @ 3500 rpm.

3. Transfer resulting solution to filament spinning apparatus as shown in FIG. 2 syringe pump reservoir. Close, attach piping; begin the filament spinning apparatus air flow and heaters. Begin solution addition. Collect fibers and place into a glass jar and seal.

### Initial Filament Spinning Apparatus Settings

| | | Electric Panel | | | | | | | |
|---|---|---|---|---|---|---|---|---|---|
| | | Temperature Settings | | | Flow Meters on Test | | | Steam | |
| | Air | Degrees Centigrade | | | Stand SCFM | | | Needle | |
| Wall | Wall Dry | | Dry | | | Dry | | Valve | Fluid |
| Att'n | #1, #2 | Att'n | Air | Dry | Att'n | Air | Dry | Turns | Flow Rate |
| psi | psi | Air | #1 | Air #2 | Air | #1 | Air #2 | Open | Ml/min |
| 20 | 30 | 65 | 300 | 300 | 3.8 | 5.8 | 5.6 | Off | 1.0-1.6 |

### Test Methods

### Viability/Count Test Method

Viability of microorganisms in web comprising a filament comprising one or more microorganisms is determined as follows.

Sample Preparation

Webs comprising filaments to be tested are removed from any protective packaging. One or more filaments to be tested are removed from the web. Filaments are tested neat (without any protective packaging such as blister packs or other similar packaging). The filaments to be tested are conditioned at 30° C.+/−2° C. and 30%+/−2% relative humidity in an open container for 28 and 56 days prior to testing and then tested immediately after the 28 or 56 days of conditioning. In addition, the filaments to be tested are conditioned at 25° C.+/−2° C. and 60%+/−2% relative humidity in an open container for 28 and 56 days prior to testing and then tested immediately after the 28 or 56 days of conditioning.

Testing Procedure

1. Dissolve 2.0 g of a filament comprising one or more microorganisms into 18 mL of a general purpose medium selected according to microorganism being tested, for example, but not limited to, sterile TSB (tryptic soy broth) (1:10 dilution) (Accumedia Manufacturers Inc. of Lansing, Mich.) in a 100 mL beaker and vortex for 10 minutes using a magnetic stirrer (Labline Model No. 1250 or equivalent) and magnetic stirring rod (5 cm) to form a concentrated microorganism suspension.

2. Make serial dilutions of the concentrated microorganism suspension from Step 1 above using TSB medium up to −8 (1:100000000).

3. Spiral Plating the serial dilutions from Step 2 above with an AutoPlate 4000 or 5000 Automated Spiral Plater from Spiral Biotech or a similar instrument is used for the plating of prepared serial dilutions on pre-poured petri plates selected according to microorganism being tested, for example, but not limited to Agar gel petri plates, (20-25 mL per plate). The dilution series prepared from the above methods are each individually plated according to the standard spiral plating method known in the art in duplicate. The plater dispenses 50 μL of each serial dilution circularly across the petri plate surface.

4. Invert each plate and incubate at 35° C. (+/−2° C.) for 48 (+/−4) hours to 72 hours, under anaerobic conditions in an anaerobe chamber or anaerobe box or aerobic conditions in an aerobic chamber or aerobic box depending on what microorganism being tested.

5. At the end of the incubation period (Step 4 above) remove each plate and count CFUs using Q-Count from Spiral Biotech.

6. The total count (total level) of a microorganism present in the filament (CFU/gram of filament) is calculated based on weight of the filament used, the CFU count of the microorganism obtained from the Q-Count software, and the dilution factor if the Q-Count software doesn't automatically factor in the dilution factor.

The log loss value is calculated as the difference between the final count of microorganisms (CFU/gram of filament) and the initial count of microorganisms added to the filament-forming composition that produced the filament (CFU/gram of filament). If the initial count of microorganisms is not known to the tester, then the difference between the final count of microorganisms (CFU/gram of filament) conditioned at 30° C.+/−2° C. and 30%+/−2% relative humidity for 28 days prior to testing and the final count of microorganisms (CFU/gram of filament) conditioned at 23° C.+/−2.2° C. and 50%+/−10% relative humidity for 2 hours (an estimate of the initial count until the actual initial count is obtained).

### Dissolution Test Method

Apparatus and Materials

ViTiny VT300 Model VT-300 Digital Portable Microscope (manufactured by ViTiny USA) or equivalent;

25 mm×75 mm microscope slides (Gold Seal® Products Rite-On® Micro Slides Catalog No. 3050, Gold Seal® Products, Portsmouth, N.H.)

22 mm×22 mm microscope cover glass (Corning Labware Cover Glass No. 1 CS2000 Number 2845-22)

Condition sealed filament samples (previously in freezer) at 23° C.+/−2.2° C. and 50%+/−10% relative humidity for at least 2 hours prior to testing. If the filament samples are in the form of a bundle of filaments, use tweezers to pull off a small amount of the filaments (70-100 mg) from the bundle. Use a second tweezer to tease apart the small amount of filaments so that individual filaments are easily distinguished. Place the teased apart filaments on a microscope slide. Cover the teased apart filaments on the slide with a cover glass positioning it so that filaments are near the cover glass edge so that upon addition of water, quick and unimpeded water flow occurs over the filaments. The filaments are under a loading of about 3.4×10⁻⁴ g/mm² as a result of the cover glass being positioned on the filaments. Focus the microscope on the filaments near the cover glass edge. Begin a video recording of the filaments. Using a disposable transfer pipette, apply de-ionized water (200-250 mg) to the edge of the cover glass. Begin timer. When filaments are dissolved, end timer and record time of dissolution. The timing can be done by replaying the recorded video later. Three replicates of each filament sample are run and the average dissolution time is reported to within ±5 seconds. Average dissolution time is in units of seconds.

### Diameter Test Method

The average diameter of a discrete filament or a filament within a nonwoven web or film is determined by using a Scanning Electron Microscope (SEM) or an Optical Microscope and an image analysis software. A magnification of 200 to 10,000 times is chosen such that the filaments are suitably enlarged for measurement. When using the SEM, the samples are sputtered with gold or a palladium compound to avoid electric charging and vibrations of the filament in the electron beam. A manual procedure for determining the filament diameters is used from the image (on monitor screen) taken with the SEM or the optical microscope. Using a mouse and a cursor tool, the edge of a randomly selected filament is sought and then measured across its width (i.e., perpendicular to filament direction at that point) to the other edge of the filament. A scaled and calibrated image analysis tool provides the scaling to get actual reading in μm. For filaments within a nonwoven web or film, several filament are randomly selected across the sample of the nonwoven web or film using the SEM or the optical microscope. At least two portions the nonwoven web or film (or web inside a product) are cut and tested in this manner. Altogether at least 90 such measurements are made and then all data are recorded for statistical analysis. The recorded data are used to calculate average (mean) of the filament diameters, standard deviation of the filament diameters, and median of the filament diameters.

### Dissolution Test Method

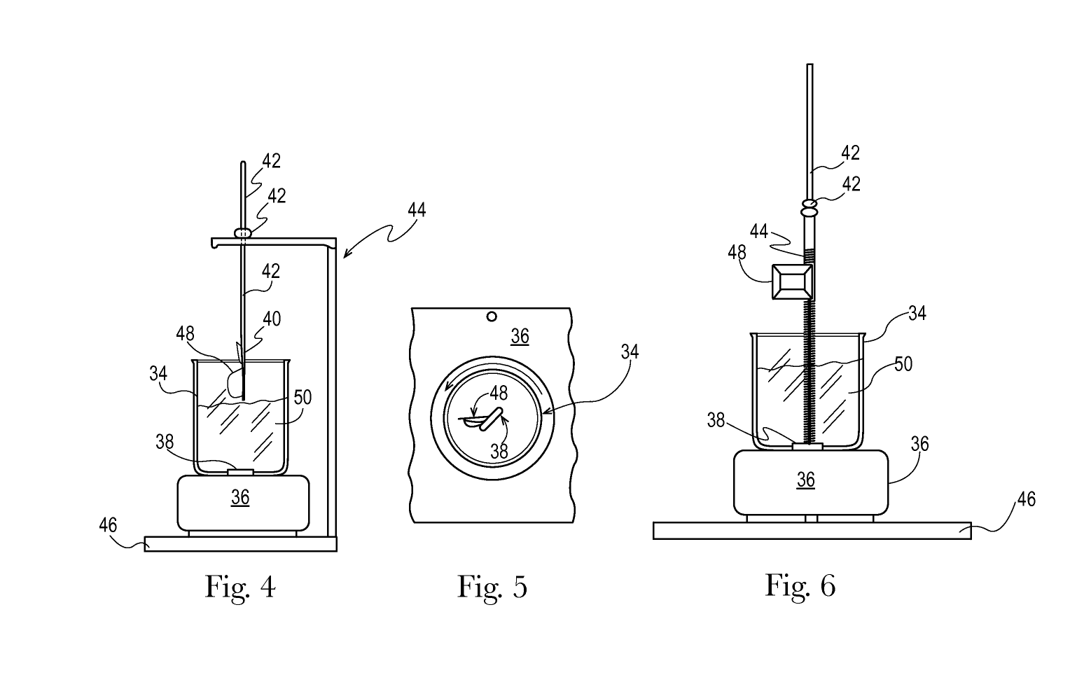

Apparatus and Materials (also, see FIGS. 4-6):

600 mL Beaker 34

Magnetic Stirrer 36 (Labline Model No. 1250 or equivalent)

Magnetic Stirring Rod 38 (5 cm)

Thermometer (1 to 100° C.+/−1° C.)

Cutting Die—Stainless Steel cutting die with dimensions 3.8 cm×3.2 cm

Timer (accurate to at least 0.1 second)

Alligator clamp (about one inch long) 40

Depth adjuster rod 42 and holder 44 with base 46

Polaroid 35 mm Slide Mount (commercially available from Polaroid Corporation or equivalent) and 35 mm Slide Mount Holder (or equivalent) 48

Deionized water (equilibrated at 23° C.±1° C.) 50

Test Protocol

Equilibrate web samples in constant temperature and humidity environment of 23° C.±1° C. and 50% RH±2% for at least 2 hours.

Measure the basis weight of a web sample to be tested using Basis Weight Method defined herein.

Cut three dissolution test specimens from the web sample using cutting die (3.8 cm×3.2 cm), so it fits within the 35 mm slide mount 48 which has an open area dimensions 24×36 mm.

Lock each specimen in a separate 35 mm slide mount 48.

Place magnetic stirring rod 38 into the 600 mL beaker 34.

Fill beaker 34 with 500 mL±5 mL of the deionized water 50.

Place full beaker 34 on magnetic stirrer 36, turn on stirrer 36, and adjust stir speed until a vortex develops and the bottom of the vortex is at the 400 mL mark on the beaker 34.

A trial run may be necessary to ensure the depth adjuster rod 42 is set up properly. Secure the 35 mm slide mount 48 in the alligator clamp 40 of the 35 mm slide mount holder such that the long end of the slide mount is parallel to the water surface. The alligator clamp 40 should be positioned in the middle of the long end of the slide mount. The alligator claim 40 is soldiered to the end of a depth adjuster rod 42. The depth adjuster rod 42 is set up in a way, so that when the slide mount 48 is lowered into the water, the entire specimen is completely submerged in the water at the center of the beaker 34, the top of the specimen is at the bottom of the vortex, and the bottom of the slide mount/slide mount holder is not in direct contact with the stirring rod 38. The depth adjuster rod 42 and alligator clamp 40 should be set so that the position of the specimen's surface is perpendicular to the flow of the water.

In one motion, drop the secured slide and clamp into the water and start the timer. The specimen is dropped so that the specimen is centered in the beaker. Disintegration occurs when the specimen breaks apart. Record this as the disintegration time. When all of the visible specimen is released from the slide mount, raise the slide out of the water while continuing the monitor the solution for undissolved specimen fragments. Dissolution occurs when all specimen fragments are no longer visible. Record this as the dissolution time.

Three replicates of each web sample are run and the average disintegration and dissolution times are recorded. Average disintegration and dissolution times are in units of seconds.

The average disintegration and dissolution times are normalized for basis weight by dividing each by the sample basis weight as determined by the Basis Weight Method defined herein. Basis weight normalized disintegration and dissolution times are in units of seconds/gsm of sample (s/(g/m²)).

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A filament comprising one or more filament-forming materials and one or more microorganisms, wherein at least one of the microorganisms exhibits less than a 2.7 log viability loss after being exposed to 30° C./30% RH conditions for 28 days as measured according to the Viability/Count Test Method.

2. The filament according to claim 1 wherein at least one of the microorganisms is releasable from the filament when exposed to conditions of intended use.

3. The filament according to claim 1 wherein the at least one microorganism is selected from the group consisting of: prokaryotes, eukaryotes, viruses, bacteriophages, and mixtures thereof.

4. The filament according to claim 1 wherein at least one of the microorganisms comprises a probiotic.

5. The filament according to claim 1 wherein at least one of the microorganisms comprises a labile microorganism.

6. The filament according to claim 1 wherein the filament-forming material is selected from the group consisting of: polyvinyl alcohol, polyvinyl alcohol derivatives, polyethylene oxide, starch, starch derivatives, cellulose, cellulose derivatives, carboxymethyl cellulose, hydroxypropylmethyl cellulose, hydroxyethyl cellulose, hydroxypropyl cellulose, polyvinyl pyrrolidone, sodium alginate, xanthan gum, tragacanth gum, guar gum, acacia gum, Arabic gum, polyacrylic acid, methylmethacrylate copolymer, carboxyvinyl polymer, chitosan, chitosan derivatives, polyethylene glycol, hemicellulose, hemicelluloses derivatives, polyacrylamide, and copolymers and mixtures thereof.

7. The filament according to claim 1 wherein the filament further comprises a stabilizing agent.

8. The filament according to claim 1 wherein the filament exhibits an average Dissolution Time of less than 60 minutes as measured according to the Dissolution Test Method.

9. The filament according to claim 1 wherein the filament exhibits an average diameter of greater than 1 μm as measured according to the Diameter Test Method.

10. The filament according to claim 1 wherein the filament contains at least 10³ CFU/g on a dry filament basis of at least one of the microorganisms after being exposed to 30° C./30% RH conditions for 28 days as measured according to the Viability/Count Test Method.

11. The filament according to claim 1 wherein the filament comprises a bicomponent filament.

12. A disposable absorbent article comprising one or more filaments according to claim 1.

13. The disposable adsorbent article according to claim 15 wherein the disposable absorbent article is selected from the group consisting of: feminine hygiene pads, pantiliners, tampons, sanitary napkins, adult incontinence pads, adult incontinence pants, diapers, baby pants, toddler pants, overnight pants, swim pants, and mixtures thereof.

14. A filament comprising one or more filament-forming materials and one or more microorganisms, wherein at least one of the microorganisms exhibits less than a 6.5 log viability loss after being exposed to 30° C./30% RH conditions for 56 days as measured according to the Viability/Count Test Method.

15. The filament according to claim 14 wherein at least one of the microorganisms is releasable from the filament when exposed to conditions of intended use.

16. The filament according to claim 14 wherein at least one of the microorganisms comprises a probiotic.

17. The filament according to claim 14 wherein at least one of the microorganisms comprises a labile microorganism.

18. The filament according to claim 14 wherein the filament-forming material is selected from the group consisting of: polyvinyl alcohol, polyvinyl alcohol derivatives, polyethylene oxide, starch, starch derivatives, cellulose, cellulose derivatives, carboxymethyl cellulose, hydroxypropylmethyl cellulose, hydroxyethyl cellulose, hydroxypropyl cellulose, polyvinyl pyrrolidone, sodium alginate, xanthan gum, tragacanth gum, guar gum, acacia gum, Arabic gum, polyacrylic acid, methylmethacrylate copolymer, carboxyvinyl polymer, chitosan, chitosan derivatives, polyethylene glycol, hemicellulose, hemicelluloses derivatives, polyacrylamide, and copolymers and mixtures thereof.

19. A disposable absorbent article comprising one or more filaments according to claim 14.

20. The disposable adsorbent article according to claim 19 wherein the disposable absorbent article is selected from the group consisting of: feminine hygiene pads, pantiliners, tampons, sanitary napkins, adult incontinence pads, adult incontinence pants, diapers, baby pants, toddler pants, overnight pants, swim pants, and mixtures thereof.
